(19) United States
(12) Patent Application Publication
(10) Pub. No.: US 2006/0057581 A1
(43) Pub. Date: Mar. 16, 2006

(54) Microfabricated fluidic device for fragmentation

(75) Inventors: Frank Karlsen, Klokkarstua (NO); Jan Lichtenberg, Neuchatel (CH); Elisabeth Verpoorte, Neuchatel (CH)

Correspondence Address: SENNIGER POWERS, ONE METROPOLITAN SQUARE, 16TH FLOOR, ST LOUIS, MO, 63102, US

(73) Assignee: Norchip AS, Klokkarstua (NO)

(21) Appl. No.: 10/533,479

(22) PCT Filed: Nov. 3, 2003
(86) PCT No.: PCT/GB03/04768

§ 371 Date: Apr. 29, 2005

(30) Foreign Application Priority Data
Nov. 1, 2002 (GB) 0225542.0

(51) Int. Cl. C12Q 1/68; C12M 1/34

(52) U.S. Cl. 435/6; 435/287.2

(57) ABSTRACT

A microfabricated device for fragmenting nucleic acids present in a fluid sample, the device comprising an inlet port (20), a fragmentation cell (1), and an outlet port (10) downstream from said inlet port (20), said cell (1) being in fluid communication with said ports, and wherein said outlet port is dimensioned to impede the flow of a fluid sample out of said cell (1) so as to effect shearing of nucleic acids molecules therein.

The present invention relates to a microfluidic device for nucleic acid fragmentation. The device may be used in or conjunction with a microfabricated reaction chamber system for carrying out, for example, a nucleic acid sequence amplification and detection process on a nucleic acid sample.

Random fragmentation of DNA or RNA is often necessary as a sample pretreatment step for, for example, nucleic acid analysis or genomic library generation. Fragmentation may be achieved biochemically using restriction enzymes, or through application of a physical force to break the molecules (see, for example, P. N. Hengen, Trends in Biochem. Sci., vol. 22, pp. 273-274, 1997 and P. F. Davison, Proc. Nat. Acad. Sci. USA, vol. 45, pp. 1560-1568, 1959).

DNA fragmentation by shearing usually involves passing the sample through a short constriction. Assuming a constant volumetric flow rate, the flow velocity increases rapidly at the constriction inlet. The coiled DNA molecules unfurl as a result, straightening out along the direction of flow. Whether a stretched molecule actually breaks in half or depends not only on flow rate, but also on molecule length and constriction cross-section. DNA can be sheared by pushing it through a syringe needle. A more complex instrument relies on an HPLC pump to push a sample repeatedly through a closed loop containing an orifice, with 10 or more cycles yielding fragments as small as 300 bp.

By the term microfabricated device or system as used herein is meant any device manufactured using processes that are typically, but not exclusively, used for batch production of semiconductor microelectronic devices, and in recent years, for the production of semiconductor micromechanical devices. Such microfabrication technologies include, for example, epitaxial growth (eg vapour phase, liquid phase, molecular beam, metal organic chemical vapour deposition), lithography (eg photo-, electron beam-, x-ray, ion beam-), etching (eg chemical, gas phase, plasma), electrodeposition, sputtering, diffusion doping and ion implantation. Although non-crystalline materials such as glass may be used, microfabricated devices are typically formed on crystalline semiconductor substrates such as silicon or gallium arsenide, with the advantage that electronic circuitry may be integrated into the system by the use of conventional integrated circuit fabrication techniques. Combinations of a microfabricated component with one or more other elements such as a glass plate or a complementary microfabricated element are frequently used and intended to fall within the scope of the term microfabricated used herein. Also intended to fall within the scope of the term microfabricated are polymeric replicas made from, for example, a crystalline semiconductor substrate.

The isolation and purification of DNA and/or RNA from bacterial cells and virus particles is a key step in many areas of technology such as, for example, diagnostics, environmental monitoring, forensics and molecular biology research.

Microfabrication is an attractive construction method for producing devices for carrying out biological processes for which very small sample volumes are desirable, such as DNA sequence analysis and detection.

One such device, for carrying out a polymerase chain reaction (PCR) followed by a detection step is disclosed in U.S. Pat. No. 5,674,742. Lamb wave pumps are used to transport DNA primers, polymerase reagents and nucleotide reagents from three separate storage chambers into a single reaction chamber as and when required to carry out a PCR process, with the temperature of the reaction chamber being cycled as required.

Another microfabricated device, for carrying out a chemical reaction step followed by an electrophoresis separation step, is disclosed in Analytical Chemistry 1994, 66, 4127-4132. Etched structures in a silicon substrate covered by a glass plate provide a reaction chamber and connections to buffer, analyte, reagent and analyte waste reservoirs, as well as an electrophoresis column connected to a waste reservoir.

Nucleic acid sequence-based amplification (NASBA) is a primer-dependent technology that can be used for the continuous amplification of nucleic acids in a single mixture at one temperature (isothermal nucleic acid amplification method) and was one of the first RNA transcription-based amplification methods described. NASBA normally offers a simple and rapid alternative to PCR for nucleic acid amplification, and is capable of yielding an RNA amplification of a billion fold in 90 minutes. With respect to other amplification systems such as the PCR technique, the ability of NASBA to homogeneously and isothermally amplify RNA analytes extends its application range from viral diagnostics to the indication of biological activities such as gene expression and cell viability. NASBA technology is discussed, for example, in Nature volume 350 pages 91 and 92. Nucleic acid amplification in NASBA is accomplished by the concerted enzyme activities of AMV reverse transcriptase, Rnase H, and T7 RNA polymerase, together with a primer pair, resulting in the accumulation of mainly single-stranded RNA that can readily be used for detection by hybridization methods. The application of an internal RNA standard to NASBA results in a quantitative nucleic acid detection method with a dynamic range of four logs but which needed six amplification reactions per quantification. This method is improved dramatically by the application of multiple, distinguishable, internal RNA standards added in different amounts and by electrochemiluminesence (ECL) detection technology. This one-tube quantitative (Q) NASBA needs only one step of the amplification process per quantification and enables the addition of the internal standards to the clinical sample in a lysis buffer prior to the actual isolation of the nucleic acid. This approach has the advantage that the nucleic acid isolation efficiency has no influence on the outcome of the quantitation, which in contrast to methods in which the internal standards are mixed with the wild-type nucleic acid after its isolation from the clinical sample. Quantitative NASBA is discussed in Nucleic Acid Research (1998) volume 26, pages 2150-2155. Post-NASBA product detection, however, can still be a labour-intensive procedure, normally involving enzymatic bead-based detection and electrochemiluminescent (ECL) detection or fluorescent correlation spectrophotometry. However, as these methodologies are heterogeneous or they require some handling of sample or robotic devices that are currently not cost-effective they are relatively little used for high-throughput applications. A homogeneous procedure in which product detection is concurrent with target amplification by the generation of a target-specific signal would facilitate large-scale screening and full automation. Recently, a novel nucleic acid detection technology, based on probes (molecular beacons) that fluoresce only upon hybridization with their target, has been introduced.

Fluidics is the science of liquid flow in, for example, tubes. For microfabricated devices, flow of a fluid through the one or more sets of micro or nano sized reaction chambers is typically achieved using a pump such as a syringe, rotary pump or precharged vacuum or pressure source external to the device. Alternatively, a micro pump or vacuum chamber, or lamb wave pumping elements may be provided as part of the device itself. Other combinations of flow control elements including pumps, valves and precharged vacuum and pressure chambers may be used to control the flow of fluids through the reaction chambers. Other mechanisms for transporting fluids within the system include electro-osmotic flow. The accurate manipulation of nanolitre volumes of a fluid sample using such techniques can be problematic and complicated.

The present invention seeks to address at least some of the problems of the prior art.

The present invention provides a microfabricated device for fragmenting nucleic acids present in a fluid sample, the device comprising an inlet port, a fragmentation cell, and an outlet port downstream from said inlet port, said cell being in fluid communication with said ports, and wherein said outlet port is dimensioned to impede the flow of a fluid sample out of said cell so as to effect shearing of nucleic acids molecules therein.

DNA and/or RNA breaks under mechanical force when pumped through a narrow orifice, due to rapid stretching of the molecule. A pressure-driven flow can lead to a shear force, which leads to fragmentation of the nucleic acids. DNA and/or RNA typically breaks in the middle of the strand. Repetitive shearing results in smaller fragments. The final fragment size will depends on a number of factors including the flow rate, the orifice cross-section (at the inlet and outlet), the number of shearing steps per run, and the initial DNA/RNA length.

Advantageously, the width of the fragmentation cell abruptly decreases at the outlet port. This achieves a high velocity gradient in the region adjacent the outlet port.

The outlet port typically comprises a constriction, preferably having a width in the range of from 1 to 100 μm, more preferably from 5 to 50 μm.

The fragmentation cell typically comprises a chamber having a bottom wall in which is formed the outlet port, the bottom wall being generally perpendicular to the direction of flow of fluid through the outlet port. The outlet port is preferably formed in approximately the middle (i.e. the mid-point of the width) of the bottom wall.

In a preferred embodiment, the fragmentation cell has the shape of an irregular polygon (preferably an irregular hexagon) with an essentially straight bottom wall in which the outlet port is formed at approximately the mid point (i.e. the mid-point of the width), and wherein the bottom wall is substantially perpendicular to the longitudinal axis of the outlet port. In this case, the bottom wall is typically adjacent and substantially perpendicular to two lower side wall portions. The upper portions of the side walls preferably taper inwardly to meet the inlet port.

The fragmentation cell will typically have a top wall in which the inlet port is formed, and side walls which extend from the top wall to the bottom wall. The side walls may taper inwardly to meet the inlet port and/or may taper inwardly to meet the outlet port.

The side walls portions next to or adjacent the inlet port advantageously subtend an angle of less than 90 degrees to the longitudinal axis of the inlet port. Such a gradual opening allows for substantially bubble-free filling of the cell.

In a preferred embodiment of the microfabricated device, the fragmentation cell is generally pear shaped with an essentially straight bottom wall in which the outlet port is formed at approximately the mid point (i.e. the mid-point of the width), the bottom wall being substantially perpendicular to the longitudinal axis of the outlet, and wherein the bottom wall is connected by curved walls to side walls, which converge or taper inwardly to meet the inlet port.

The device preferably further comprises an obstacle located in the cell in the direct path between the inlet and outlet ports. In this case, the space between sides of the obstacle and sides of the cell preferably defines a bifurcated path for the fluid sample. Advantageously, the obstacle is shaped so that the flow path of a fluid sample in a region adjacent the outlet port is substantially perpendicular to the longitudinal axis of the outlet. In other words, the direction of fluid flow in the region of the cell just prior to the outlet is preferably substantially perpendicular to the longitudinal axis of the outlet (and typically also the longitudinal axis of the inlet).

The obstacle may be in the form of a generally triangular obstacle, with its three sides substantially parallel to the bottom wall and side walls of the cell, the space between the sides of the obstacle and the sides of the cell defining a bifurcated path for the fluid sample.

The fragmentation cell will typically be asymmetric about the horizontal axis and substantially symmetric about the longitudinal axis, the longitudinal axis being essentially coincident with the direction of flow.

The microfabricated device preferably further comprises an access channel in fluid communication with the inlet port. The access channel typically connects the inlet port to the source of the fluid sample and preferably has a greater flow area than the inlet port. A sample loading chamber may also be provided to facilitate controlled loading of a sample into the fragmentation cell.

The microfabricated device preferably further comprises collection means in fluid communication with the outlet port for collecting the fragmented nuclei acids (contained in the fluid sample).

The microfabricated device preferably further comprises mean for effecting flow of a sample into the inlet port, through the fragmentation cell and out of the outlet port. Such means may comprise one or more pumps. Alternatively, such means may comprises one or more variable volume chambers in communication with the inlet port and/or outlet port, wherein altering the volume of the variable volume chamber(s) effects and/or restricts flow of a fluid sample into and/or out of the fragmentation cell. The variable volume chamber typically comprises a flexible membrane overlying a hollow recess in the substrate.

The microfabricated device will typically comprise a substrate and an overlying cover, the fragmentation cell being defined by a recess in a surface of the substrate and the adjacent surface of the cover. The substrate may be formed from silicon, for example, and the overlying cover from glass, for example. In this case, the glass cover is preferably anodically bonded to the silicon substrate, optionally through an intermediate silicon oxide layer formed on the surface of the substrate.

The microfabricated device preferably comprises at least first and second fragmentation cells, the outlet port of the first cell being in fluid communication with the inlet port of the second cell. Of course, in this manner, a third fragmentation cell may be provided, the outlet port of the second cell being in fluid communication with the inlet port of the third cell. Likewise, fourth, fifth, sixth, etc cells may be provided. Advantageously, the microfabricated device comprises a plurality (at least two) of serially connected fragmentation cells. Microfluidic implementation allows linear sequences of multiple fragmentation cells for repetitive shearing in one run. The size of the outlet port preferably decreases the further down stream the fragmentation cell. Thus, the size of the outlet port may progressively decrease from the first fragmentation cell to the last fragmentation cell (downstream). For example, the orifice size of the outlet ports can be reduced as degree of fragmentation increases (eg 10 constrictions from 50 down to 5 μm).

A modified design of the fragmentation cell includes an island or obstacle located in the chamber between the inlet and outlet ports. In other words, an obstacle is placed in the path of the jet between the inlet and outlet. Flow is then forced to follow paths around the obstacle, which prevents jets and circulatory flow from appearing. This improves the flow pattern for better shearing and bifurcated flow may be achieved. Furthermore, it has been found that rounded corners in the design of the cell improve the homogeneity of the flow pattern when compared to structures containing sharp corners.

The design of the fragmentation cell is preferable such so as to achieve a substantially perfect laminar, sink-type flow. It has been found that this type of flow is advantageous for good nucleic acid fragmentation performance.

The microfabricated device may be used for fragmenting nucleic acids present in a biological fluid, a dairy product, an environmental fluid or drinking water.

The present invention also provides a microfabricated reaction chamber system for carrying out a nucleic acid sequence amplification and detection process on a nucleic acid sample, the system comprising a microfabricated device as herein described. The device and process as herein described and according to the present invention may also be used to fragment polymers, such as polysaccharides, and proteins which may be present in a fluid sample.

The present invention also provides an apparatus for the analysis of biological and/or environmental samples, the apparatus comprising a device or a system as herein described. The apparatus may be disposable.

The present invention also provides an assay kit for the analysis of biological and/or environmental samples, the kit comprising a device or a system as herein described and means for contacting the sample with the device. The assay kit may be disposable.

The present invention also provides a method for DNA and/or RNA fragmentation based on the application of a hydrodynamic shear force to the DNA/RNA molecules, using a device as herein described. The device mimics mesoscopic fragmentation devices (such as described in P. J. Oefner, S. P. Hunicke-Smith, L. Chiang, F. Dietrich, J. Mulligan, and R. W. Davis, Nucleic Acids Research, vol. 24, pp. 3879-3886, 1996), taking advantage of microfluidic assets such as low dead-volume and short processing times. The device enables a 48 kbp lambda DNA sample to be fragmented down to 4 kbp.

Fragmentation may be achieved by passing a sample through alternating, horizontal contractions of the flow path from, say, 100 μm down to 30 μm or smaller. Generally speaking, this layout requires only one lithography and etching step, and is therefore easily fabricated. If larger diameter orifices are incorporated into the channels, in order to prevent clogging, then higher flow rates must be used in order to obtain the same shearing rates. Sequences of progressively shrinking orifices ensure a low risk of clogging at the beginning (approx. 50 μm widths), and higher shear rates towards the end of the channel to achieve smaller fragments (orifices approx. 10 μm wide). Structures having series of orifices of decreasing diameter help alleviate any clogging problems. This is because the larger fragments will have been broken into smaller fragments by the time they arrive at smaller diameter orifices.

The microfabricated device may be used in a system for carrying out any suitable biological or chemical reaction such as, for example, enzyme reactions, immuno reactions, sequencing, hybridisation.

The nucleic acid sample may be derived from, for example, a biological fluid, a dairy product, an environmental fluids and/or drinking water. Examples include blood, serum, saliva, urine, milk, drinking water, marine water and pond water. For many complicated biological samples such as, for example, blood and milk, it will be appreciated that before one can isolate and purify DNA and/or RNA from bacterial cells and virus particles in a sample, it is first necessary to separate the virus particles and bacterial cells from the other particles in sample. It will also be appreciated that it may be necessary to perform additional sample preparation steps in order to concentrate the bacterial cells and virus particles, i.e. to reduce the volume of starting material, before proceeding to break down the bacterial cell wall or virus protein coating and isolate nucleic acids. This is important when the starting material consists of a large volume, for example an aqueous solution containing relatively few bacterial cells or virus particles. This type of starting material is commonly encountered in environmental testing applications such as the routine monitoring of bacterial contamination in drinking water.

As indicate earlier, the device and process as herein described may be used to fragment polymers, such as polysaccharides, and proteins which may be present in a fluid sample such as a biological fluid, a dairy product, an environmental fluids and/or drinking water.

The system is preferably designed to cater for a sample volume of =<50 nl, preferably =<20 nl, more preferably =<10 nl. Thus the volume of each of the reaction chambers will typically be =<50 nl, preferably =<20 nl, more preferably <=10 nl. However, and as will be appreciated, larger sized chambers may be used, for example chambers having a volume of 100 to 500 nl.

An integrated microfabricated reaction chamber system may be provided with a plurality of devices as described above, each of which may have a separate outlet port. In this way a range of different analysis processes may be carried out simultaneously within a single micromachined device.

The present invention also provides a method for the manufacture of a microfabricated device as herein described, which method comprises:
(i) providing a substrate having at least one recess in a surface thereof;
(ii) providing a cover; and
(iii) bonding the cover to the substrate to create at least one fragmentation cell defined by said at least one recess in said surface of the substrate and the adjacent surface of the cover. The substrate may be formed from silicon, for example, and the overlying cover from glass, for example. In this case, the glass cover is preferably anodically bonded to the silicon substrate, optionally through an intermediate silicon oxide layer formed on the surface of the substrate. The recess in the silicon may be formed using reactive-ion etching. Other materials such as polymeric materials may also be used for the substrate and/or cover.

Preferably, and in particular if optical observations of the contents of the cell are required, the overlying cover is made of an optically transparent substance or material, such as glass or Pyrex.

The term recess as used herein is also intended to cover a variety of features including, for example, grooves, slots, holes, trenches and channels, including portions thereof.

The system or at least a master version thereof will typically be formed from or comprise a semiconductor material, although dielectric (eg glass, fused silica, quartz, polymeric materials and ceramic materials) and/or metallic materials may also be used. Examples of semiconductor materials include one or more of: Group IV elements (i.e. silicon and germanium); Group III-V compounds (eg gallium arsenide, gallium phosphide, gallium antimonide, indium phosphide, indium arsenide, aluminium arsenide and aluminium antimonide); Group II-VI compounds (eg cadmium sulphide, cadmium selenide, zinc sulphide, zinc selenide); and Group IV-VI compounds (eg lead sulphide, lead selenide, lead telluride, tin telluride). Silicon and gallium arsenide are preferred semiconductor materials. The system may be fabricated using conventional processes associated traditionally with batch production of semiconductor microelectronic devices, and in recent years, the production of semiconductor micromechanical devices. Such microfabrication technologies include, for example, epitaxial growth (eg vapour phase, liquid phase, molecular beam, metal organic chemical vapour deposition), lithography (eg photo-, electron beam-, x-ray, ion beam-), etching (eg chemical, gas phase, plasma), electrodeposition, sputtering, diffusion doping, ion implantation and micromachining. Non-crystalline materials such as glass and polymeric materials may also be used.

Examples of polymeric materials include PMMA (Polymethyl methylacrylate), COC (Cyclo olefin copolymer), polyethylene, polypropylene, PL (Polylactide), PBT (Polybutylene terephthalate) and PSU (Polysulfone), including blends of two or more thereof. Hot embossing of such polymeric materials may be used to form the device.

Combinations of a microfabricated component with one or more other elements such as a glass plate or a complementary microfabricated element are frequently used and intended to fall within the scope of the term microfabricated used herein.

The substrate base may be provided with a coating of thickness typically up to 1 μm, preferably less than 0.5 μm. The coating is preferably formed from one or more of the group comprising polyethylene glycol (PEG), Bovine Serum Albumin (BSA), tweens and dextrans. Preferred dextrans are those having a molecular weight of 9,000 to 200,000, especially preferably having a molecular weight of 20,000 to 100,000, particularly 25,000 to 75,000, for example 35,000 to 65,000). Tweens (or polyoxyethylene sorbitans) may be any available from the Sigma Aldrich Company. PEGs are preferred as the coating means, either singly or in combination. By PEG is embraced pure polyethylene glycol, i.e. a formula HO—(CH₂CH₂O)ₙ—H wherein n is an integer whereby to afford a PEG having molecular weight of from typically 200-10,000, especially PEG 1,000 to 5,000; or chemically modified PEG wherein one or more ethylene glycol oligomers are connected by way of homobifunctional groups such as, for example, phosphate moieties or aromatic spacers. Particularly preferred are polyethylene glycols known as FK108 (a polyethylene glycol chain connected to another through a phosphate); and the PEG sold by the Sigma Aldrich Company as product P2263. The above coatings applied to the surfaces of the cell/chamber, inlets, outlets, and/or channels can improve fluid flow through the system. In particular, it has been found that the sample is less likely to adhere or stick to such surfaces. PEG coatings are preferred.

The device/system will typically be integrally formed. The device/system may be microfabricated on a common substrate material, for example a semiconductor material as herein described, although a dielectric substrate material such as, for example, glass or a ceramic material could be used. The common substrate material is, however, preferably a plastic or polymeric material and suitable examples are given above. The system may preferably be formed by replication of, for example, a silicon master. This may be achieved, for example, by hot embossing of a polymeric material.

The microfabricated device/system may be designed to be disposable after it has been used once or for a limited number of times. This is an important feature because it reduces the risk of contamination.

The microfabricated device/system may be incorporated into an apparatus for the analysis of, for example, biological fluids, dairy products, environmental fluids and/or drinking water. Again, the apparatus may be designed to be disposable after it has been used once or for a limited number of times.

The microfabricated system/apparatus may be included in an assay kit for the analysis of, for example, biological fluids, dairy products, environmental fluids and/or drinking water, the kit further comprising means for contacting the sample with the device. Again, the assay kit may be designed to be disposable after it has been used once or for a limited number of times.

The microfabricated system as herein described is also intended to encompass nanofabricated devices.

For a silicon or semiconductor master, it is possible to define by, for example, etching or micromachining, one or more of variable volume chambers, microfluidic channels, reaction chambers and fluid interconnects in the silicon substrate with accurate microscale dimensions (deep reactive-ion etching (DRIE) is a preferred technique). A plastic replica may then be made of the silicon master. In this manner, a plastic substrate with an etched or machined microstructure may be bonded by any suitable means (for example using an adhesive or by heating) to a cover thereby forming the enclosed fragmentation cell(s), inlet(s), outlet(s) and connecting channel(s).

The present invention may also be used in conjunction with a microfabricated device for nucleic acid extraction, which device preferably comprises an extraction cell having an inlet port for introducing a sample (eg from the fragmentation cell) and an outlet port downstream from said inlet port for withdrawing extracted nuclei acids, wherein said extraction cell is at least partially filled with silica beads or particles. Such a device may be used for nucleic acid extraction. This aspect of the present invention is based to an extent on the finding that NA binds to silica surfaces in the presence of chaotropic agents The extraction cell may have any suitable shape and configuration but will typically be in the form of a channel or a chamber. The extraction cell preferably further comprises one or more sets of electrodes adjacent the silica beads or particles for collecting and/or preconcentrating the eluted nucleic acids. Said one or more sets of electrodes preferably comprise platinum electrodes. Means may therefore be provided for applying a potential difference accross the electrodes. The integration of electrodes may be used to reversibly collect and preconcentrate the eluted NA on-chip. Thus, this aspect of the present invention enables combined nucleic acid extraction and enrichment to be achieved.

The optional microfabricated device for nucleic acid extraction will typically comprise a substrate and an overlying cover, the extraction cell being defined by a recess in a surface of the substrate and the adjacent surface of the cover. The substrate is preferably formed from silicon or poly(dimethylsiloxane) (PDMS). PDMS channels may be fabricated by replica molding using a 2-layer SU-8 master. Dry, 15-35 μm silica particles can be packed into the channels using a vacuum. Platinum electrodes can be patterned on a Pyrex wafer by a lift-off process.

The present invention also provides a process for fragmenting nucleic acids present in a fluid sample, the process comprising:
(a) providing a microfabricated device as herein described;
(b) providing a fluid sample comprising nucleic acids;
(c) injecting the fluid sample into the inlet port of said device, through the fragmentation cell and out of the outlet port; and
(d) collecting the thus fragmented sample at the outlet port.

The process preferably further involves a nucleic acid sequence amplification and detection process on the fragmented nucleic acid sample.

The present invention will now be described, by way of example, with reference to the accompanying drawings, of which:—

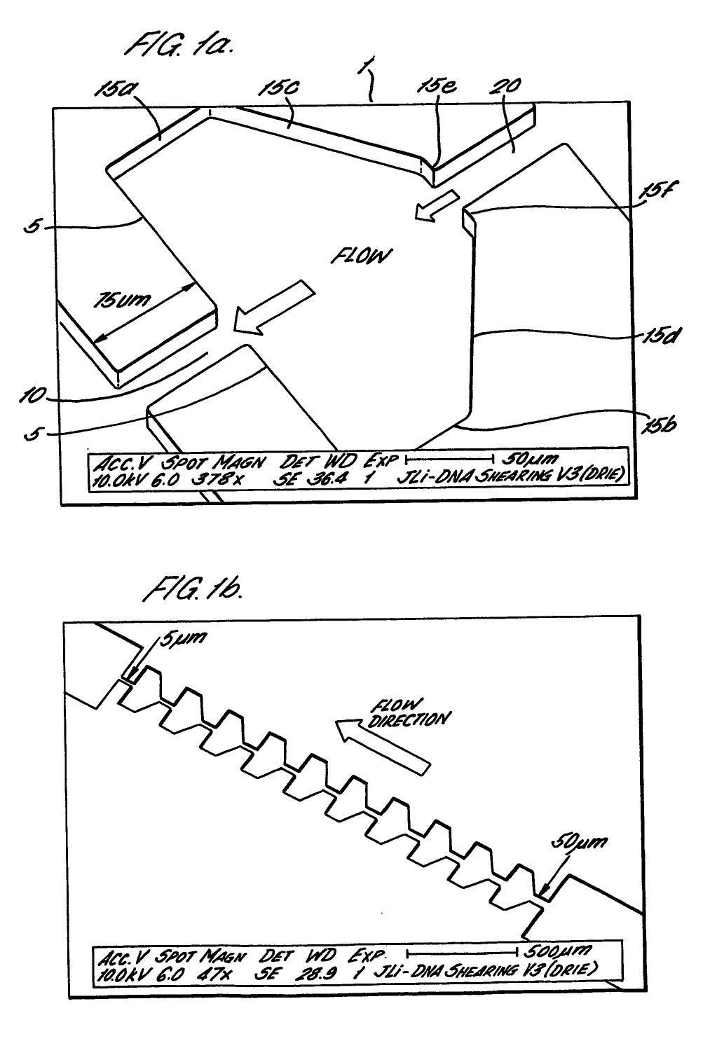

FIGS. 1(a) and (b) are close-up views of a microfabricated device according to the present invention formed from silicon.

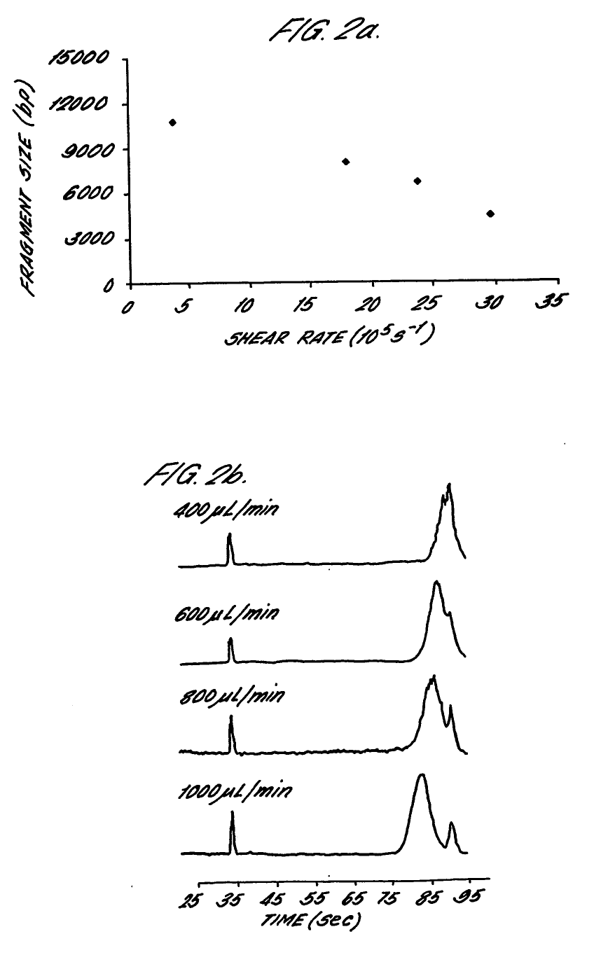

FIGS. 2(a) and (b) shows the dependence of fragment size on shear rate for a chip with 10 constrictions of approx. 15 μm width.

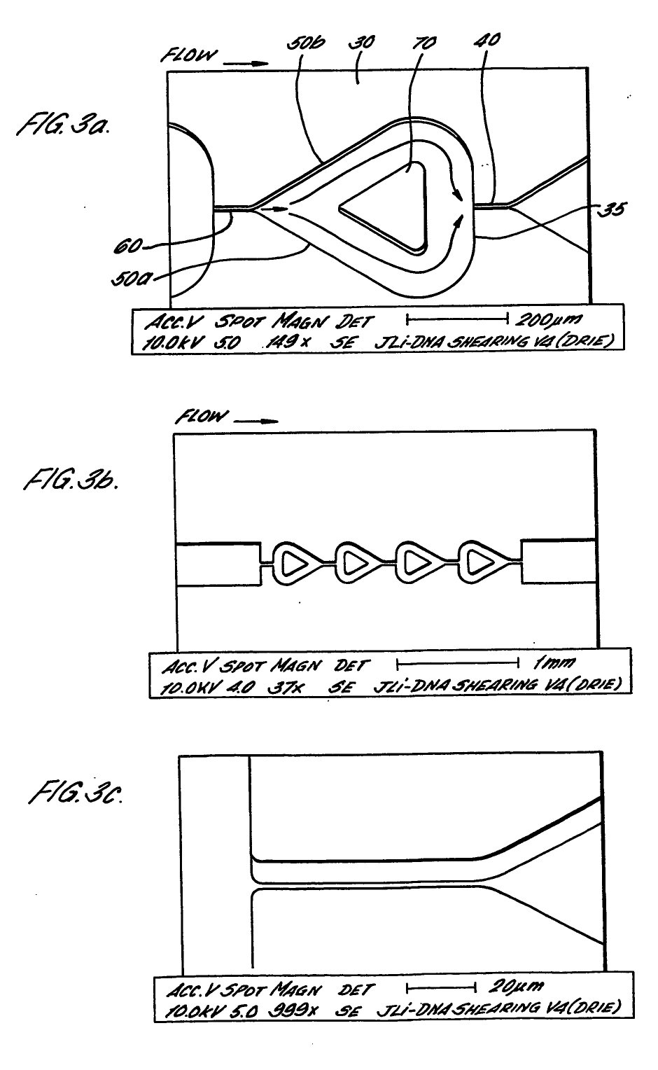

FIGS. 3 (a), (b) and (c) shows an alternative structure for the fragmentation chamber in which an island or obstacle is inserted in the chamber to effect bifurcated flow. (a) Close-up of cell with approx. 10 μm wide orifices, the wide of the channels are approx. 100 μm, and the flow direction is from left to right. (b) View of a series of device with five orifices, each approx. 25 μm wide. To the left and right, approx. 400 μm wide access channels are visible. (c) Close-up of a approx. 10 μm wide orifice.

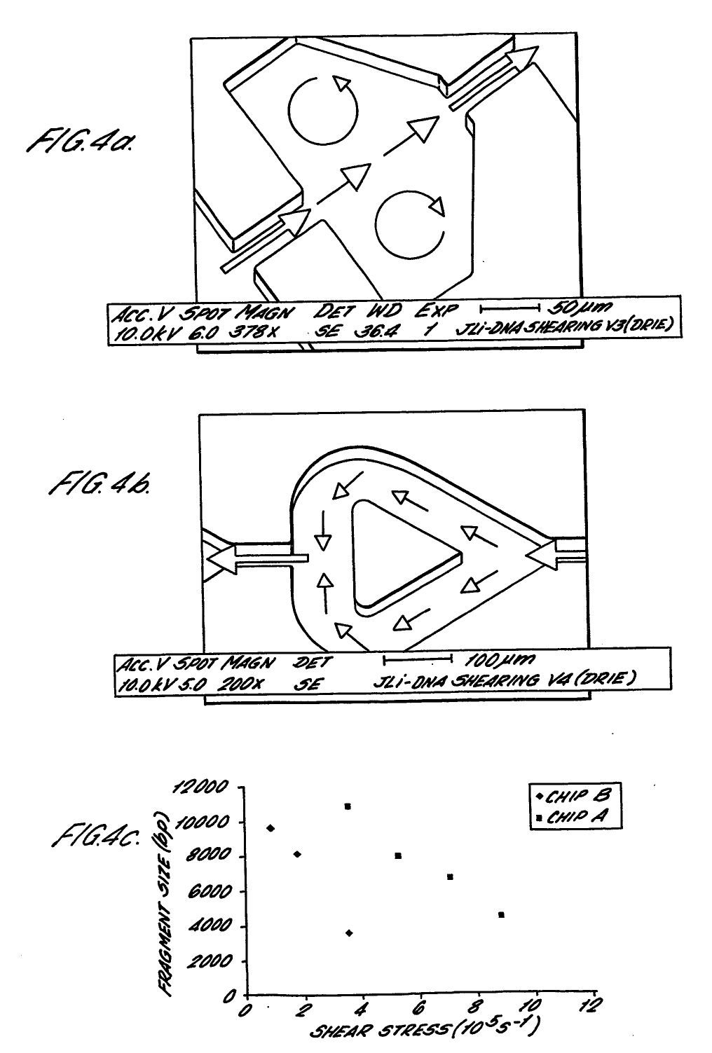

FIGS. 4 (a), (b) and (c) provide a comparison between the fragmentation chambers according to FIG. 1 (a) and FIG. 3 (a). FIG. 4(a) corresponds to a cell similar to the one shown in FIG. 1(a). FIG. 4(b) corresponds to a cell similar to the one shown in FIG. 3(a). FIG. 4(c) is a plot of fragment size against shear rate for the two structures.

FIGS. 1(a) and (b) show a close-up view of one fragmentation cell (or shearing unit) in a microfabricated silicon device, and a series of consecutive cells with monotonically decreasing orifices, respectively.

FIG. 1 (a) shows a scanning electron micrograph (SEM) of a fragmentation cell 1 (or shearing unit), one in a connected series made by deep reactive-ion etching (DRIE) in silicon. The constriction outlet 10 (approx. 75 μm long) is designed to have an abrupt change in cross-section from large to small in the flow direction. At the chamber inlet 20, a gradual opening has been found to help avoid air bubbles being trapped in the structure. The constriction width (i.e. the width of the outlet and inlet) is approx. 25 μm and the feature depths approx. 50 μm.

The shape of fragmentation cell 1 may be described as an irregular hexagon with an essentially straight bottom wall 5 in which the outlet 10 is formed at approximately the mid point. It can be seen that the bottom wall 5 is substantially perpendicular to the longitudinal axis of the outlet 10 (and the direction of flow). Thus, the bottom wall 5 subtends an angle of approximately 90 degrees to the longitudinal axis of the outlet 10 (and the direction of flow). The bottom wall 5 is adjacent and substantially perpendicular to two lower side wall portions 15a and 15b. The upper portions 15c and 15d of the side walls taper inwardly to meet the inlet 20 at the top of the cell 1. Thus, upper side walls portions 15c and 15d each subtend an angle of less than 90 degrees to the longitudinal axis of the inlet (and the direction of flow). It can be seen, however, that the uppermost side wall portions 15e and 15f immediately adjacent the inlet 20 subtend an angle of approximately 90 degrees to the longitudinal axis of the inlet 10 (and the direction of flow). It can also be seen that the cell 1 is asymmetric about the horizontal axis and substantially symmetric about the longitudinal axis (the longitudinal axis is essentially coincident with the direction of flow).

FIG. 1 (b) shows a SEM of the device with 10 constrictions with decreasing widths from approximately 50 to 5 μm. Feature depths were either approximately 50 or 75 μm. This is an example of a device with a series of constrictions of progressively decreasing width. Device clogging is reduced, since the larger DNA fragments passing through the wider constrictions at the beginning are reduced in size by the time they reach the smaller constrictions. At these later, smaller channels, the higher shear rates required for breaking the smaller fragments can be generated.

The microfabricated device will generally be part of a chip. The device comprises a substrate with the desired microstructure formed in its upper surface. The substrate may be silicon, for example, or a plastic substrate formed by replication of a silicon master. The substrate is bonded at its upper surface to a cover, thereby defining a series of fragmentation cells, inlets, outlets, and channels. The cover may be formed from plastic or glass, for example. The cover is preferably transparent and this allows observation of the fluid. In general, the device is preferably fabricated by deep reactive-ion etching (DRIE) of silicon for high aspect ratio constrictions, followed by anodic bonding of a glass cover. For example, fabrication of the devices shown in the Figures involved the following steps: (1) silicon substrate; (2) negative photoresist (eg MAN 420, approx. 2.7 μm thick) defining desired microstructure; (3) deep reactive-ion etching (approx. 50-75 μm deep); (4) photoresist removal; (5) thermal oxidation (200 nm); and (6) anodic bonding to a drilled Pyrex 7740 glass cover plate (approx. 800 V, 360° C.).

With reference to FIGS. 2(a) and (b), a syringe pump was used to pump sample (48 kbp lambda DNA (67 mg/mL in 10 mM Tris-HCl/5 mM NaCl/0.1 mM EDTA, pH 7.0)) through the chips. The electropherograms in FIG. 2(a) show that there is a shift towards smaller fragment sizes as flow rate is increased, as expected. In fact, the sharp peaks at about 35 and 90 seconds are due to size marker DNA molecules corresponding to 50 and 17000 bp, respectively. For a shear rate of 3×10⁶ s⁻¹ (1000 mL/min), fragment size could be reduced to about 4000 bp (value at the electropherogram peak maximum). A size distribution of 1370-9780 bp was obtained at this flow rate, which is somewhat larger than the 2-fold size distribution expected if fragments had been broken ideally at their midpoints each time. FIG. 2(b) gives fragment size at peak maximum as a function of shear rate, and clearly shows the decrease in DNA fragment size as shear rate increases.

FIG. 3 shows an alternative structure for the shearing chamber which sees the insertion of an island or obstacle in the chamber (approximately in the centre) to effect bifurcated flow. In other words, an obstacle is placed in the path of the jet between the inlet and outlet. Flow is then forced to follow paths around the obstacle, which prevents jets and circulatory flow from appearing. Furthermore, the rounded corners of the design have been found to improve the homogeneity of the flow pattern when compared to structures containing sharp corners. The etch depth is approximately 50 μm. The design achieves a substantially perfect laminar, sink-type flow. It has been found that this type of flow is advantageous for good nucleic acid fragmentation performance.

Comparing FIG. 3 to FIG. 1, the shape of the fragmentation cell 30 may be described as pear shaped with an essentially straight bottom wall 35 in which the outlet 40 is formed at approximately the mid point. It can be seen that the bottom wall 35 is substantially perpendicular to the longitudinal axis of the outlet 40 (and the direction of flow). Thus, the bottom wall 35 subtends an angle of approximately 90 degrees to the longitudinal axis of the outlet 40 (and the direction of flow). The bottom wall 35 is connected by curved walls 45a and 45b to side walls 50a and 50b, which taper inwardly to meet the inlet 60 at the top of the cell. Thus, the side walls 50a and 50b each subtend an angle of less than 90 degrees to the longitudinal axis of the inlet (and the direction of flow). A generally triangular obstacle 70 is located in the cell, with its three sides substantially parallel to the bottom wall 35 and side walls 50a and 50b. The space between the sides of the obstacle and the sides of the cell defines a bifurcated path for the fluid sample.

FIGS. 4 (a), (b) and (c) provide a comparison between a fragmentation chamber design based on FIG. 1 (a) and one based on FIG. 3 (a) (FIG. 4(a) corresponds to a cell similar to the one shown in FIG. 1(a), while FIG. 4(b) corresponds to a cell similar to the one shown in FIG. 3(a)). FIG. 4(c) gives fragment size at peak maximum as a function of shear rate, and clearly shows the decrease in DNA fragment size as shear rate increases, and also shows the difference in flow dynamics between the two structures results in different fragmentation performance. In particular, the design shown in FIG. 4(b) results in smaller fragments compared with the design shown in FIG. 4(a).

The present invention also provides a device which includes a microfabricated device/system as herein described, together and preferably in fluid communication with one or more of:
(A) means for filtering a sample prior to carrying out the method according to the present invention, for example to substantially remove particles contained in the sample which are larger in size that bacteria particles; and/or
(B) means for separating virus particles and/or bacterial cells from the other particles in a sample prior to carrying out the method according to the present invention; and/or
(C) means for concentrating bacterial cells and/or virus particles, i.e. to reduce the volume of starting material, prior to carrying out the method according to the present invention; and/or
(D) means for breaking down the bacterial cell wall or virus protein coating and isolate nucleic acids prior to carrying out the method according to the present invention.

A microfabricated device embodying the invention may form an integral part of a larger microfabricated analysis device constructed as a single unit and containing, for example, apparatus for carrying out various sample preparation steps, and containing the various reagents required to carry out the sample preparation steps. Such a microfabricated analysis device could contain some or all of the control and data analysis circuitry required for its operation.

### EXAMPLE

General

Chips were fitted into chip holders where polyetherketone (PEEK) tubing could be connected to the microchannels in the Si-device. The fragmentation of the DNA/RNA was done manually using a Hamilton-syringe. Before fragmentation the device was flushed with 100 μl pure water (Sigma W4502) to remove any air in the system. The fragmentation was done by pumping 100 μl of a HPV 45 positive sample. The sample was pretreated as follows: 100 μl HPV 45 positive sample in methanol-buffer (PreservCyt, CYTYC) was sentrifugated for 5 min at 800 rpm. The pellet was then resuspended in 900 μl water (Sigma). 100 μl of this solution was then used in the fragmentation device.

Specification of the Fragmentation Device

A device as herein described and having a fragmentation cell of the type shown in FIG. 4(a) was used. The orifices width (or constriction) is approximately 20 μm, while the orifice length is approximately 50 μm.

Experimental Procedure:

1. Pump 100 μl pure water through the device to remove any air (Sigma W4502, RNase- and DNase free).
2. Pump 100 μl of the sample.
3. Pump 100 μl of RNa-later, non-diluted (Ambion 7022).
4. Collect sample (extract) and the RNa-later and make analysis (NASBA).
   Results: NASBA (Nucleic Acid Sequence Based Amplification)

A positive centrifugated HPV 45 sample was pumped through the fragmentation device and the outcome was analysed in NASBA the same day. The sample-extract was tested non-diluted with PreTect HPV Proofer kit (NorChip). The NASBA-result showed a 6.14 times increase in the signal for HPV 45 from the startlevel. Negative control (pure Sigma-water) showed no increase in signal for HPV 45. Positive control for HPV 45 (artificial designed HPV 45 DNA-oligo, diluted 1 to a million) showed 6.99 times increase in signal.

Results: RNase-Activity

The sample extract was also tested for RNase-activity with the RNase Alert Lab Test Kit (Ambion 1964). The sample extract showed no Rnase activity compared to a negative and positive RNase control.

Implementation of DNA shearing in microfluidic devices is advantageous for a number of reasons. Microchannel geometries incorporating small (=<50 μm), highly reproducible constrictions are possible, allowing generation of high shear rates at comparatively low flow rates (in the order of μL/min rather than mL/min). This, combined with smaller device volumes, facilitates processing of small samples. Multiple shearing steps can be accomplished by inserting a number of constrictions into a microchannel. In this way, sample does not need to be continually recirculated through the same constriction, and flow system dead volume can be substantially reduced.

1-39. (canceled)

40. A microfabricated device for fragmenting nucleic acids present in a fluid sample, the device comprising an inlet port, a fragmentation cell, and an outlet port downstream from said inlet port, said cell being in fluid communication with said ports, and wherein said outlet port is dimensioned to impede the flow of a fluid sample out of said cell so as to effect shearing of nucleic acids molecules therein, wherein the fragmentation cell comprises a chamber having a bottom wall in which is formed the outlet port, the bottom wall being generally perpendicular to the direction of flow of fluid through the outlet port, and wherein the fragmentation cell has a top wall in which the inlet port is formed, and side walls which extend from the top wall to the bottom wall, and wherein the side walls taper inwardly to meet the inlet port.

41. A microfabricated device as claimed in claim 1, wherein the fragmentation cell has the shape of an irregular polygon, preferably an irregular hexagon, with an essentially straight bottom wall in which the outlet port is formed at approximately the mid point, and wherein the bottom wall is substantially perpendicular to the longitudinal axis of the outlet port.

42. A microfabricated device as claimed in claim 1, wherein the fragmentation cell is generally pear shaped with an essentially straight bottom wall in which the outlet port is formed at approximately the mid point, the bottom wall being substantially perpendicular to the longitudinal axis of the outlet, and wherein the bottom wall is connected by curved walls to side walls, which converge or taper inwardly to meet the inlet port.

43. A microfabricated device as claimed in claim 1, wherein the width of the fragmentation cell abruptly decreases at the outlet port.

44. A microfabricated device as claimed in claim 1, wherein the outlet port comprises a constriction, preferably having a width in the range of from 1 to 100 μm, more preferably from 5 to 50 μm.

45. A microfabricated device as claimed in claim 1, wherein the outlet port is formed in approximately the middle of the bottom wall.

46. A microfabricated device as claimed in claim 1, wherein the side walls taper inwardly to meet the outlet port.

47. A microfabricated device as claimed in claim 1, wherein the bottom wall is adjacent and substantially perpendicular to two lower side wall portions.

48. A microfabricated device as claimed in claim 8, wherein the upper portions of the side walls taper inwardly to meet the inlet port.

49. A microfabricated device as claimed in claim 1, wherein side walls or portions thereof next to or adjacent the inlet port subtend an angle of less than 90 degrees to the longitudinal axis of the inlet port.

50. A microfabricated device as claimed in claim 1, wherein the fragmentation cell comprises a bottom wall in which the outlet port is formed at approximately the mid point, the bottom wall being substantially perpendicular to the longitudinal axis of the outlet, and side walls which converge or taper inwardly to meet the inlet port.

51. A microfabricated device as claimed in claim 1, wherein the device further comprises an obstacle located in the cell in the direct path between the inlet and outlet ports.

52. A microfabricated device as claimed in claim 12, wherein the space between sides of the obstacle and sides of the cell defines a bifurcated path for the fluid sample.

53. A microfabricated device as claimed in claim 12, wherein the obstacle is shaped so that the flow path of a fluid sample in a region adjacent the outlet port is substantially perpendicular to the longitudinal axis of the outlet.

54. A microfabricated device as claimed in claim 12, wherein the obstacle is in the form of a generally triangular obstacle, with its three sides substantially parallel to the bottom wall and side walls of the cell, the space between the sides of the obstacle and the sides of the cell defining a bifurcated path for the fluid sample.

55. A microfabricated device as claimed in claim 1, wherein the fragmentation cell is asymmetric about the horizontal axis and substantially symmetric about the longitudinal axis, the longitudinal axis being essentially coincident with the direction of flow.

56. A microfabricated device as claimed in claim 1, further comprising an access channel in fluid communication with the inlet port.

57. A microfabricated device as claimed in claim 1, further comprising collection means in fluid communication with the outlet port.

58. A microfabricated device as claimed in claim 1, further comprising means for effecting flow of a sample into the inlet port, through the fragmentation cell and out of the outlet port.

59. A microfabricated device as claimed in claim 19, wherein said means for effecting flow comprises one or more pumps.

60. A microfabricated device as claimed in claim 19, wherein said means for effecting flow comprises one or more variable volume chambers in communication with the inlet port and/or outlet port, wherein altering the volume of the variable volume chamber(s) effects and/or restricts flow of a fluid sample into and/or out of the fragmentation cell.

61. A microfabricated device as claimed in claim 1 which comprises a substrate and an overlying cover, the fragmentation cell being defined by a recess in a surface of the substrate and the adjacent surface of the cover.

62. A microfabricated device as claimed in claim 22, wherein the substrate is formed from silicon and the overlying cover from glass.

63. A microfabricated device as claimed in claim 23, wherein the glass cover is anodically bonded to the silicon substrate, optionally through an intermediate silicon oxide layer formed on the surface of the substrata.

64. A microfabricated device as claimed in claim 1 which comprises at least first and second fragmentation cells, the outlet port of the first cell being in fluid communication with the inlet port of the second cell.

65. A microfabricated device as claimed in claim 25, further comprising a third fragmentation cell, the outlet port of the second cell being in fluid communication with the inlet port of the third cell.

66. A microfabricated device as claimed in claim 25 comprising a plurality of serially connected fragmentation cells.

67. A microfabricated device as claimed in claim 25, wherein the size of the outlet port decreases the further down stream the fragmentation cell.

68. A microfabricated device as claimed in claim 28, wherein the size of the outlet port gradually decreases from the first fragmentation cell to the last fragmentation cell downstream.

69. A microfabricated device as claimed in claim 1 for fragmenting nucleic acids present in a biological fluid, a dairy product, an environmental fluid or drinking water.

70. A microfabricated reaction chamber system for carrying out a nucleic acid sequence amplification and detection process on a nucleic acid sample, the system comprising a microfabricated device as defined in claim 1.

71. An apparatus for the analysis of biological and/or environmental samples, the apparatus comprising a device as defined in claim 1.

72. An assay kit for the analysis of biological and/or environmental samples, the kit comprising a device as defined in any one of claim 1 and means for contacting the sample with the device.

73. An apparatus as claimed in claim 32 which is disposable.

74. A process for fragmenting nucleic acids present in a fluid sample, the process comprising:
(a) providing a device as defined in claim 1;
(b) providing a fluid sample comprising nucleic acids;
(c) pumping the fluid sample into the inlet port of said device, through the fragmentation cell and out of the outlet port; and
(d) collecting the thus fragmented sample at the outlet port.

75. A process as claimed in claim 35 which further involves a nucleic acid sequence amplification and detection process on the fragmented nucleic acid sample.
